(19) United States
(12) Patent Application Publication
(10) Pub. No.: US 2023/0071742 A1
(43) Pub. Date: Mar. 9, 2023

(54) METHOD AND SYSTEM FOR ADVERSARY RESILIENT SCREENING OF SYNTHETIC GENE ORDERS

(71) Applicant: B.G. NEGEV TECHNOLOGIES AND APPLICATIONS LTD., AT BEN-GURION UNIVERSITY, Beer Sheva (IL)

(72) Inventors: Rami PUZIS, Ashdod (IL); Dor FARBIASH, Omer (IL)

(21) Appl. No.: 17/904,675

(22) PCT Filed: Feb. 17, 2021
(86) PCT No.: PCT/IL2021/050186

§ 371 Date: Aug. 19, 2022

Related U.S. Application Data
(60) Provisional application No. 62/978,840, filed on Feb. 20, 2020.

(51) Int. Cl. G16B 30/10; G16B 50/30; G06F 16/903

(52) U.S. Cl. CPC G16B 30/10; G16B 50/30; G06F 16/90335

(57) ABSTRACT

A system and methods for adversary resilient screening of synthetic gene orders, include: applying a first alignment algorithm to generate multiple local alignments of a query sequence with a target sequence, wherein each local alignment aligns a substring of the query sequence to a substring of the target sequence to maximize an alignment score; determining unaligned sections of the query sequence to be alignment gaps; and removing k largest alignment gaps of the query sequence to generate a clean query sequence.

### FIELD OF THE INVENTION

The present invention generally relates to the field of genetic sequencing and sequence analysis.

### BACKGROUND

Synthetic biology is an emerging bioengineering technology that plays a significant role in personalized medicine and pharmaceutical manufacturing. Today arbitrary synthetic DNA can be ordered online and delivered within several days. However, synthetic DNA manufacturing may also be employed to generate dangerous substances that may be subsequently used for biological attacks. Consequently, most synthetic gene providers employ sequence search techniques to screen DNA orders.

Screening of DNA orders typically follows protocols to screen for possible toxins, pathogens, and other biological agents that pose a significant threat to public health and safety, agents that are collectively referred to as sequences of concern (SOCs). The U.S. Department of Health & Human Services has issued a “Screening Framework Guidance for Providers of Synthetic Double-Stranded DNA,” a protocol meant to reduce the risk of generating SOCs. Similar protocols include the Harmonized Screening Protocol v2.0 (HSP), employed by the International Gene Synthesis Consortium (IGSC) and the International Association Synthetic Biology (IASB) Code of Conduct for Best Practices in Gene Synthesis. US regulation also defines items on the Commerce Control List as sequences of concern.

The HHS guidelines recommend using a sequence alignment tool, such as BLAST (“Basic Local Alignment Search Tool”), to compare gene orders with known sequences in the GenBank database. BLAST is one of many algorithms developed for aligning sequences of nucleotides or amino acids. The BLAST algorithm is described in Altschul, et al., “Gapped BLAST and psi-BLAST: A New Generation of Protein Database Search Programs,” Nucleic Acids Research, 25(17):3389-3402, 1997, incorporated herein by reference. BLAST operates by matching n-grams—short sequences of letters (words)—and extending these matches to form local alignments between the sequences.

GenoTHREAT is a software package implementing screening according to the HHS guidelines, and described by Adam, et al., in “Correspondence: Strengths and limitations of the federal guidance on synthetic DNA, Nature Biotechnology, 29(3):208-210, 2011, which is incorporated herein by reference. GenoTHREAT partitions query sequences into 200 base pair (bp) fragments before screening against known SOCs. Another software package, BlackWatch, also for screening against toxic agents, is described by Jones in “Sequence Screening,” in Epstein, et al. editors, Working Papers for Synthetic Genomics: Risks and Benefits for Science and Society, pages 1-16. 2007, incorporated herein by reference. An additional method named NNTox is described by Aashish et al., “Gene Ontology-based protein Toxicity Prediction using Neural Network,” in Scientific Reports, 9(1):1-10, 2019, incorporated herein by reference. NNTox employs machine learning to identify toxic sequences.

In addition to these targeted biosecurity tools, there are more general systems that can be used to predict the function of DNA and protein sequences. These include InterProScan described by Jones et al., in “Interproscan 5: Genome-scale Protein Function Classification,” Bioinformatics, 30(9):1236-1240, 2014, and SeqScreen, described by Dreycey et al., in “Seqscreen: A Biocuration Platform for Robust Taxonomic and Biological Process Characterization of Nucleic Acid Sequences of Interest” (2019 IEEE International Conference on Bioinformatics and Biomedicine, pages 1729-1736), both of which are incorporated herein by reference. These approaches provide detailed information for the human analyst to investigate a hit including both suspicious and legitimate sections of a DNA sequence.

However, these and similar screening measures for detecting SOCs may be insufficient to detect SOCs in synthetic DNA sequences that are hidden by methods of “genetic obfuscation.”

A sequence of concern (SoC) may be “obfuscated” by being split into small fragments. These fragments are then interleaved with legitimate (i.e., benign, non-SOC) DNA sequences that have various levels of similarity to the SOC fragments. When the resulting, merged sequence is used to scan a sequence database, the results may show a legitimate sequence as being a Best Match to the merged sequence, thus hiding the presence of the SOC.

An attacker faces two main challenges when attempting to hide an SOC by obfuscation. He must find legitimate DNA that can successfully camouflage the SOC fragments; and he must subsequently “decode,” i.e., reconstruct, the obfuscated DNA, with a minimal set of standard biological primitives. To be reconstructed, the SOC fragments embedded within camouflage DNA need to be split from the camouflage DNA and then joined with each other. Splicing and reconstructing a full SOC from the fragments is facilitated by known DNA editing processes, which may be implemented within living cells.

The “clustered regularly interspaced short palindromic repeats” (CRISPR) complex is a part of the bacterial immune system that was adapted by bioengineers to perform precise DNA editing in live biological systems. The most common DNA CRISPR-based editing system consists of a Cas9 protein and a guide RNA sequence (gRNA). The Cas9 protein performs a cut in a double stranded DNA (dsDNA) molecule at specific locations called protospacer adjacent motifs (PAMs). The gRNA contains a short replica of the region following the PAM that needs to be cut by Cas9. For gRNA creation, the DNA should contain a promoter, a copy of the gRNA target site, and a terminator; collectively these are referred to as a gRNA scaffold.

The CRISPR system thus facilitates removal of the camouflage genes between consecutive SOC fragments and the subsequent stitching of consecutive SOC fragments together, forming operational DNA.

A dsDNA that was cut by a CRISPR can repair itself. Such a repair process is error prone; however, precise repairs of cut DNA may be performed using a process known as homology directed repair (HDR). To activate HDR, a cell should contain a DNA sequence that repeats the sequence of nucleotides to the left and right of the cut point (left and right arms of the HDR template respectively) and a small number of nucleotides that can be inserted between them at the cut point. Using CRISPR and HDR it is possible to remove long DNA fragments. (It is also possible to replace long fragments, a process known as knock-in.)

Biological attacks may then be executed with the rejoined SOC fragments. The potential for such attacks underlines the need to harden the synthetic DNA supply chain with protections against cyber-biological threats.

### SUMMARY

Embodiments of the present invention provide a system and methods for adversary resilient screening of synthetic gene orders, to detect obfuscated sequences of concern (SOCs). Steps of a process provided by these embodiments may include: receiving a query sequence for screening; applying a first alignment algorithm to generate multiple local alignments of the query sequence with a target sequence, wherein each local alignment aligns a substring of the query sequence to a substring of the target sequence to maximize an alignment score; from the aligned substrings of the query sequence, determining unaligned sections of the query sequence to be alignment gaps; and removing k largest alignment gaps of the query sequence to output a clean query sequence.

In some embodiments, a number P of aligned substrings of the query sequence, when combined, align with more of the target sequence than any other combination of aligned substrings, and wherein k is set as equal to P -1.

Additionally, the steps may include applying a second alignment algorithm to generate a clean alignment between the clean query sequence and the target sequence and to generate a respective clean alignment score, as well as to output the clean alignment score as an indicator of an SOC homology of the query sequence.

In some embodiments, the first and second alignment algorithms are the same alignment algorithm. The first and second alignment algorithms may be BLAST algorithms

In some embodiments, an adjusted clean alignment score may be calculated as the clean alignment score less a gap removal penalty proportional to k. The clean alignment and the adjusted clean alignment score may be output as indicators of an SOC homology of the query sequence. The adjusted clean alignment score may also be a function of a probability of successful gap removal to generate an intended SOC.

In further embodiments, the gap removal penalty may be a per gap removal penalty (prm) multiplied by k.

The gap removal penalty may be a function of a number of base pairs removeable by bioengineering tools. In some embodiments, the clean alignment score may include a negative increment for each gap opening (pgo) and for each gap extension (pgx), and prm may be set equal to pgo+pgx·x, where x is the removeable number of base pairs.

Further embodiments may include removing different quantities k of gaps from the query sequence, and reapplying the alignment algorithm to each of the multiple clean query sequences to generate multiple respective clean alignments and respective adjusted clean alignment scores. Outputting the clean alignment and the adjusted clean alignment score may include determining a maximum score from among the multiple adjusted clean alignment scores and outputting the maximum score as the adjusted clean alignment score and outputting the respective clean alignment.

The target sequence may be a sequence from a database of target sequences, and the steps may include iterating generation of clean alignments and respective adjusted clean alignment scores with respect to all of the database target sequences. Embodiments may also include ordering the multiple clean alignments and the multiple adjusted clean alignment scores according to the adjusted clean alignment score. The target database may be a database of SOCs. An initial value of k may be set to be all gaps greater than a preset threshold number of base pairs, and k may be subsequently incremented by an iterative process removing the largest alignment gap not previously removed, until the adjusted clean alignment score calculated following the gap removal does not increase.

In some embodiments, the alignment score may include a positive increment for each matching character (rm) and a negative increment for each mismatching character (pmm), for each gap opening (pgo), and for each gap extension (pgx).

### BRIEF DESCRIPTION OF DRAWINGS

For a better understanding of various embodiments of the invention and to show how the same may be carried into effect, reference will now be made, by way of example, to the accompanying drawings. Structural details of the invention are shown to provide a fundamental understanding of the invention, the description, taken with the drawings, making apparent to those skilled in the art how the several forms of the invention may be embodied in practice. In the figures:

FIG. 1 is a flow chart of a process for adversary resilient screening of DNA sequences, in accordance with an embodiment of the invention; and

FIGS. 2-4 are graphs representing sequence alignment results of a process for adversary resilient screening of DNA sequences; in accordance with an embodiment of the invention.

### DETAILED DESCRIPTION

Embodiments of the present invention provide a system and methods for adversary resilient screening of synthetic gene orders, that is, screening of DNA sequences that may hide fragments of toxic or otherwise dangerous substances.

Consider a case where a bioterrorist wants to generate a synthetic virus, a dangerous toxin, or other SOCs. Screening of DNA orders is necessary to prevent such bioterrorism, but an attacker may try to circumvent screening by ordering an obfuscated SOC. Obfuscation may include multiple gRNA scaffolds, each targeting a different cut point, though this could potentially reduce the effectiveness of SOC reconstruction (“decoding”). The size of the SOC fragments and the HDR templates may also vary. Reducing the size of the SOC fragments increases their likelihood of blending within camouflage genes, but also leads to a larger number of cuts and repairs, consequently reducing the effectiveness of reconstruction. The currently known lower bound on the size of HDR templates is 64 bp.

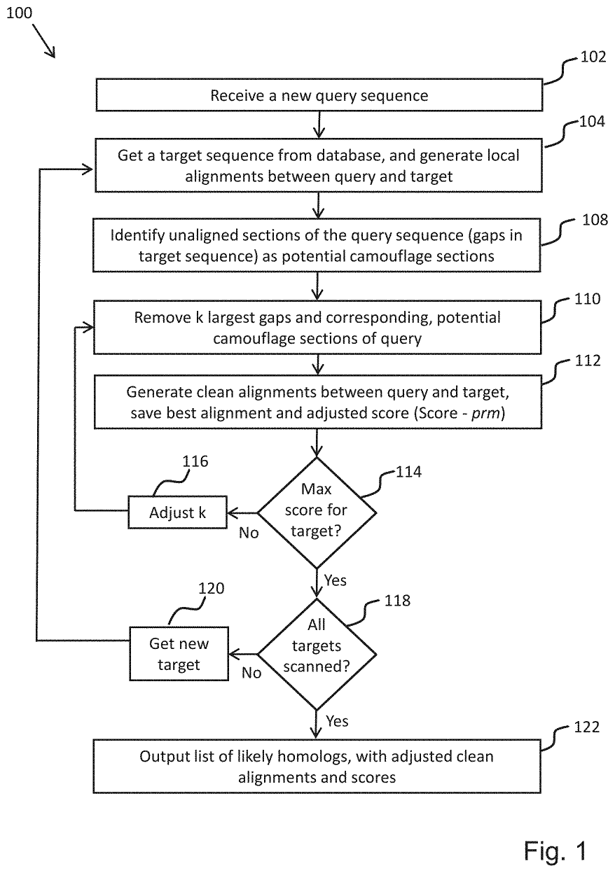

FIG. 1 is a flow chart of a DNA screening process 100 for identifying SOCs, and in particular for assessing the difficulty of SOC assembly from a given DNA sequence. The process thus improves the resilience of synthetic DNA production against “adversarial” attempts to produce SOCs. Hereinbelow, the process is also referred to as gene edit distance computation, or “GED.” GED screens a query sequence to find all substrings which are similar to fragments of an SOC. Then, GED quantifies the effort of assembling a SOC from these fragments. Although designed with a focus on SOCs, GED can quantify the effort required to assemble any target sequence t from a query sequence q using a standard CRISPR system. This determination includes a step of counting the number of cuts and repairs required for constructing the target sequence from the query sequence.

In a standard biological sequence alignment, a typical objective is to identify genes conserved in different genomes. To achieve this objective, an alignment algorithm should have parameters that include a match reward (rm), a mismatch penalty (pmm), a gap opening penalty (pgo), and a gap extension penalty (pgx).

However, identification of SOC fragments requires identification of short, conserved regions. Within these regions, alignments with target sequences should involve minimal gaps within the query sequence. On the other hand, it should be possible to concatenate multiple short, conserved regions regardless of the length of gaps that separate them. This is because when removing a sequence between two consecutive SOC fragments, using the CRISPR system and HDR templates, the distance between the fragments is irrelevant.

At a first step 102 of process 100, a query sequence q is received, which may be, for example, a sequence that is ordered for synthetic DNA production and which must be scanned to ensure that it is not an SOC.

At a step 104, the query sequence is scanned against a target sequence t, also called a subject sequence.

Alignment algorithms, such as BLAST, generally run against a database of potential target sequences and return a set of target sequences that are similar to the query sequence, as well as a set of alignments (also called ranges) for each target sequence. Algorithms based on BLAST typically compute a large set of small local alignments and extend these alignments when doing so increases the alignment score. The process described herein merges these small local alignments to maximize an adjusted score, Score^{k}.

At the step 104, an alignment algorithm generates A_{q,t} , which is a set of local alignments between q and t. Hereinbelow, q[i] denotes the i'th character in q; and t[i] denotes the i'th character in t. Every alignment in α∈A_{q,t}maps a substring (i.e., a range of character positions) in q to a substring in t, such that for any two successfully aligned character positions i>j, α(i)>α(j). Hereinbelow, a substring of q of an alignment α is denoted as dom(α) and a substring of t is denoted as img(α)=α(dom(α)). Similarly, α⁻¹ denotes the inverse alignment, such that dom(α⁻¹)=img(α) and img(α⁻¹)=dom(α).

The score of a BLAST alignment may be computed based on the number of:
matched characters, M=|{i:q[i]=t[α(i)]}|;
the number of mismatched characters, MM=|{i:q[i]≠t[α(i)]}|;
the number of gaps opened in both the query and the target sequences, G=|{i:α(i−1)≠⊥Λα(i)=⊥}|+|{i:α₋₁(i−1) ≠⊥Λα⁻¹(i)=⊥}|; and
the total extent of the gaps, GX=|{i:α(i)=⊥}|+|{i:α⁻¹(i)=⊥}|,
   where ⊥ symbolizes a character that is not aligned.

There is a reward (rm) for every matching character and penalties for mismatching characters (pmm), gap opening (pgo), and gap extension (pgx). The reward and penalties are configurable. A score of an alignment may be calculated as:

Score=rm·M−pmm·MM−pgo·G−pgx≠GX

The fraction of q characters successfully aligned is called query coverage (QC): QC(α)=MM/|q|. The percent identity is computed from the sizes of the domain, the image, and the number of successfully mapped characters:

PI(α)=2·MM/(|dom(α)|+|img(α)|)

At a step 108, unaligned sections of the query sequence (i.e., gaps in the target sequence) are identified as potential camouflage sections. A per-gap removal penalty (prm) may then be applied instead of some gap opening and extension penalties within the target sequence, for each gap removed.

At a step 110, we remove the k largest sections of potential camouflage sections. In some embodiments, the k sections removed from the query sequence may be determined as the number of disjoint local alignments (denoted as P) minus one. This output “clean” query sequence is then provided to a processing step 112 to be realigned with the same target sequence. If multiple combinations of disjoint local alignments may be applied, these combinations are also tested as described below with respect to step 116. The output clean query sequence may also be stored for subsequent analysis of adversarial attacks.

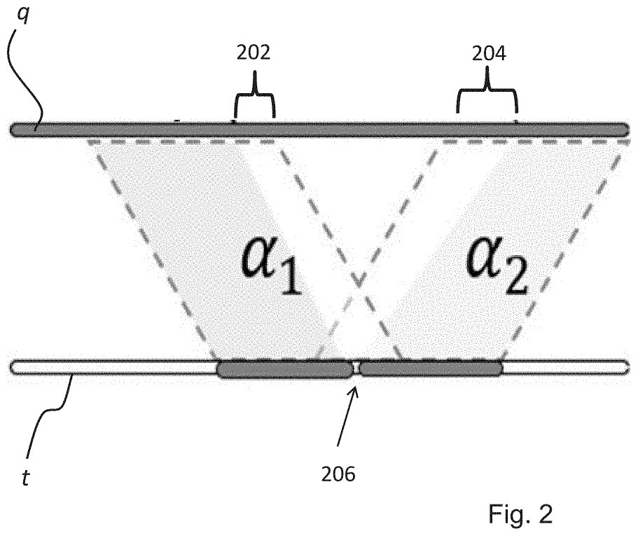

FIG. 2 indicates two local alignments of a target sequence t with disjoint sections of a query sequence, q. Note that the alignments have overlapping “images,” that is, the aligned sections of the target query overlap (the overlapping sections indicated as 202 and 204). The two sections of the query sections may be joined at a point 206 to create a clean sequence with high homology to the target sequence.

For a given target sequence gap g=[a,b] (that is, ∀i∈g, α(i)=⊥), a corresponding section g=[a,b] from the query sequence can be removed with two cuts, at a and at b respectively, followed by an HDR.

Removing the section g=[a,b] increases the score of the alignment by pgo+pgx·(b−a+1), which we then reduce by prm. G_{q}(α) and Gₜ (α) respectively represent sets of all gaps in the query and target sequences according to the alignment α. Let Gₜₖ⊆Gₜ be a subset of the k longest gaps in the target sequence to be removed. We represent a probability that all gaps in Gₜₖ will be successfully removed as γₖ. Let .start and .end denote the first and the last position, respectively, in the domain or the image of an alignment α∈A_{q,t}. Let P=α₁, . . . , αₖ be a set of alignments whose domains are disjoint, dom(αᵢ).end<dom(αᵢ₊₁).start. Their images may overlap, as depicted in FIG. 2 BLAST can find an alignment between the unified domains of the alignments (i.e., a “clean” sequence) and the target sequence. Such unified alignments are also referred to as cleaned alignments. The score of a clean alignment is indicated below as Score^{C}.

Next, we adjust the clean alignment score to account for the probability of successful removal of the Gₜₖ sections by adversarial agents. We first calculate a virtual score, Score^{V}, as a score that BLAST would produce if it were to generate the clean alignment (i.e., the alignment of the same domain and image produced by the clean alignment) but without the removal of gaps. Such a score may be calculated as follows:

$${Score}^{V} = {Score}^{C} - {\sum }_{g ∈ {G}_{tk}} ( pgo + pgx \cdot \vert g \vert )$$

That is, Score^{V} equals Score^{C}, less the gap opening and gap extension penalties, pgo and pgx, for each of the gaps removed in the clean alignment.

Next, we generate an adjusted alignment score as the result of the removal of the k longest gaps in the target sequence t, given a probability of successful gap removal γ^{|G}^{ₜₖ}^{|}, and considering the gap removal penalty prm:

$${Score}^{k} ( α ) = {Score}^{V} + {γ}^{\vert {G}_{tk} \vert} \cdot {\sum }_{g ∈ {G}_{tk}} ( pgo + pgx \cdot \vert g \vert - prm )$$

where the alignment is indicated as α; g is a subset of gaps, Gₜₖ, in the target sequence of α; the gap opening and gap extension penalties are pgo and pgx respectively; the gap removal penalty is prm; and the gap removal probability is γ. As indicated in the equation, Score^{k} is a function of a total gap removal penalty, which is a sum of gap removal penalties for all k, i.e., a penalty that is proportional to k, namely, k*prm. In addition, Score^{k} is a function of the gap removal probability, that is, the probability of successful reconstruction of the intended SOC.

In embodiments of the present invention, the match reward (rm), as well as the mismatch, gap opening, and gap extension penalties (pmm, pgo, pgx) may be set to BLAST default values. In further embodiments, parameters for a BLAST alignment may be rm=2, pmm=3, pgo=5, and pgx=2.

The value of γ may be set according to assumptions made regarding the expected biological effectiveness of an adversarial “attack” given typical bioengineering tools. A value of γ=0 signifies that no attacker could ever rely on residue Cas9 protein in the cells and the SOC obfuscation attack is impossible. A value of γ=1 signifies 100% success of gene editing, which leads to the successful reconstruction of sequences regardless of the number of SOC fragments. In one test described below, we set γ=0.99.

The gap removal penalty prm should be set to a value that would justify gap removal using bioengineering tools. For example, if only the removal of gaps larger than x bp is justified, then the gap removal penalty should be set to: prm=pgo+pgx·x.

The longer a gap is, the higher the alignment penalty and therefore the greater the score enhancement that can be achieved by removing the gap. To optimize Score^{k}, the longest gaps are therefore selected for removal. The number of gaps k that should be included in Gₜₖ to achieve the maximal Score^{k} depends on the parameters of the alignment algorithm and can be optimized using a grid search or simple hill climbing algorithm.

Referring again to FIG. 1, steps 110 through 112 are typically an iterative process, a process that is repeated until an optimal value of Score^{k} is achieved.

At a step 114, a test is made as to whether an optimal Score^{k} has been achieved. This is typically assessed by determining that increasing k for the most recent iteration has decreased the store, meaning that the highest score was achieved at the prior iteration. If different local alignments indicate different sections of the query sequence that may be preserved in the “clean alignment,” these different sections may be selected at a step 116, to be applied in iterations of steps 110 and 112 to achieve the optimal Score^{k}. Additionally, If the maximum has not been achieved, then k may be incremented at step 116, after which steps 110 and 112 are repeated. Alternatively, a set of aligned substrings of the query sequence may be selected to maximize the coverage of the target sequence. Denoting P as the number of aligned substrings in such a set, k=P−1. If multiple combinations of aligned substrings cover the same extent of the target sequence are possible, the optimal combination may be selected according to the value of Score^{k}.

Hereinbelow, a GED difference (GEDD) is a measure of the optimal number of gaps k to remove in a target sequence alignment such that the adjusted alignment score is maximized The GEDD from q to t is:

GEDD(q,t)=ARGMAXₖ(MAX_{α}Score^{k}(α)).

According to this definition Score^{GED(q,t)}(α) is the maximal adjusted score for aligning the query with a given target, after optimal removal of potential camouflage fragments. (Note that GED quantifies the process of transforming q into t but not vice versa.) Until a determination is made at a step 118 that all target sequences have been processed, steps 104 through 114 are repeated for every sequence in the target database, a new target being extracted at the database at a step 120 before each iteration. Typical the target database is a database of known SOC sequences. A final determination of the risk of a query sequence is made based on the maximal adjusted score of the query sequence for any of the SOC sequences. The maximal adjusted score corresponds to an SOC that could most directly be generated from the query sequence.

The algorithm for GED listed below shows the steps of the process 100 as heuristic pseudocode, using the symbolic representations described above:

Pseudocode: Gene Edit Distance

| | |
|---|---|
| | Input: |
| | q - a query sequence |
| | t - a target sequence |
| | Output: |
| | k - the number of cut and repair operations |
| | Score^{k} - adjusted alignment score |
| | / / List local alignments A = {_{α}} sorted by |
| | dom(α).start |
| 1 | A_{q},ₜ = BLAST(query=q, subject=t) |
| 2 | for each subset P ⊆ A_{q,t} of local alignments with disjoint |
| | domains do |
| 3 | \|_ _{αP} = MERGE(P) |
| 4 | P* = argmax_{P}Score^{\|P\|-1} (_{αP}) |
| 5 | return k = \|P*\|, Score^{k} (_{αP*}) |
| 6 | Function MERGE(_{α1},..., (_{αk}): |
| | \| /* Find global alignment between unified domains |
| | \| and t */ |
| 7 | \| A = BLAST(query = q[Sᵢdom (_{αj})],subject = t) |
| 8 | \| _{α} = cleaned global alignment unifyingA |
| | \| /* Reintroduce gaps to form a continuous |
| | \| alignment */ |
| 9 | \| _{α}U = {(i, ⊥): i ∈ [dom (_{αj}),end, dom (_{αj+} ₁).start], 1 ≤ |
| | \|_ j < k} return _{α} |

The output of GED is the adjusted alignment score and the optimal k, that is, the number of cut and repair actions required to reconstruct the target sequence from the query sequence. The adjusted alignment score quantifies both the similarity of the body of an obfuscated SOC, and the effort required to “decode” it within a cell. GED is designed to detect the main body of an obfuscated SOC, because the gRNA scaffolds and HDR templates required for splicing may be distributed among different plasmids or even different orders.

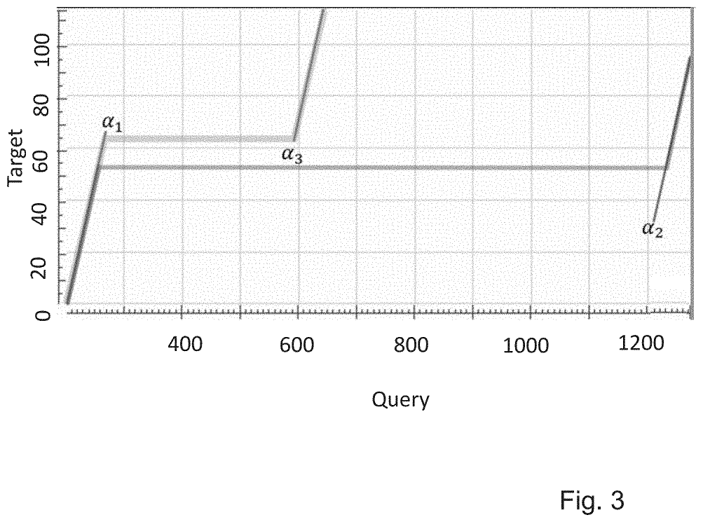

FIG. 3 presents a graph of the results of aligning an obfuscated α-conotoxin PeIA (a short, toxic pepsin) to the PeIA sequence. BLAST returns three ranges (alignments), indicated as α₁, α₂, and α₃. If pgx>0, these ranges will not be merged by BLAST due to the length of the gap that would be opened in the target sequence. Merging α₁ and α₂ would give the highest value for Score^{k} (higher than for merging α₁ and α₃, because the alignment of α₂ with the target sequence extends farther than the alignment of α₃ with the target sequence).

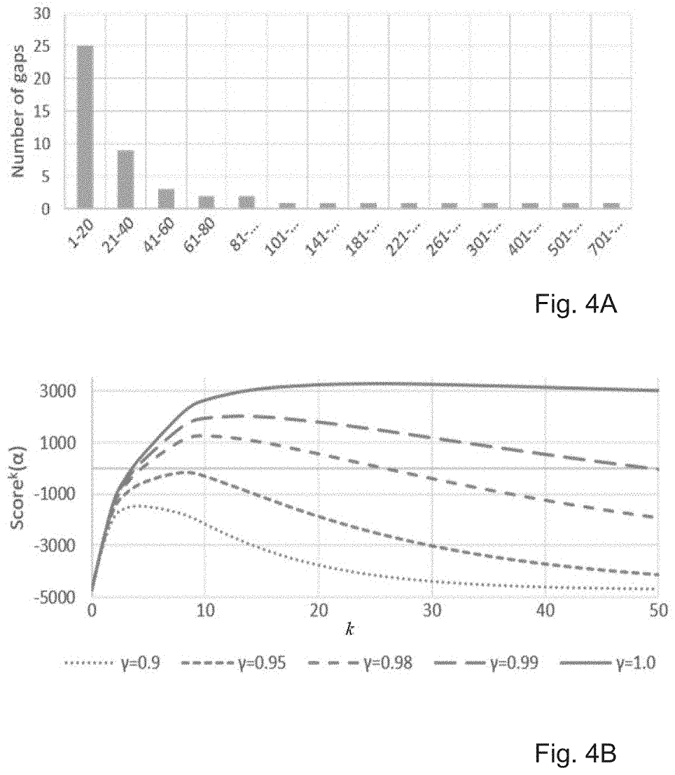

FIGS. 4A and 4B present graphs of an additional alignment example. A 10 Kpb long query sequence is aligned to a 2 Kpb long SOC. An optimal alignment between the two sequences contains 2 K matching base pairs, a few mismatches, and 50 gaps whose length is exponentially distributed, as shown in FIG. 4A. Before removing any gaps (k=0) the adjusted alignment score is less than −4,500. With a gap removal penalty of prm=20, gaps are removed, replacing gap opening and gap extension penalties with prm. FIG. 4B shows the resulting graph of k vs. the adjusted alignment score Score^{k}. As shown, with γ=0.98, k=10 results in the highest Score^{k} value. With γ=0.99, the adjusted alignment score can reach 2,000 when k=13 in this example.

Benchmark sequence screening was performed using local copies of the complete GenBank NT and NR and UniProt databases. While GenoTHREAT requires the entire database for accurate screening, GED only requires the SOCs to compare with the query sequences. We considered the true positive rate (TPR), the false negative rate (FNR), and the false positive rate (FPR). High FNR (low FPR) indicated that the screening method could be evaded using obfuscation. Because some malicious sequences are easier to detect than others, we the hit counts for each group of sequences. In order to analyze the performance of the screening algorithms, we also inspect their confidence levels. The confidence of GED (GEDConf) is simply the maximal adjusted score it returns when screening q. The value of GEDConf is between zero and one, where GEDConf=1 means that q is definitely a SOC. GED successfully detected all obfuscated malicious sequences. Moreover, the large confidence gap between the most suspicious benign sequence and the least suspicious malicious sequence showed GED's robustness as a screening algorithm. We also note that GED is an order of magnitude faster than GenoTHREAT, because it compares the query sequence with a small database of SOC and does not need to query for every 200 bp fragment.

Experiments demonstrate that 16 out of 50 obfuscated DNA samples are not detected when screened according to the HHS guidelines. We further proposed a hardened screening algorithm termed Gene Edit Distance (GED) that successfully detects all obfuscated DNA samples. Future enhancements to DNA screening may rely on machine learning for sequence analysis and DNA function prediction. Adversarial learning techniques can be used to further increase the resilience of screening algorithms against malicious DNA sequences that are not yet on the SOC list.

It is to be understood that all or part of process 100 may be implemented in digital electronic circuitry, or in computer hardware, firmware, software, or in combinations thereof. The computing system may have one or more processors and one or more network interface modules. Processors may be configured as a multi-processing or distributed processing system. Network interface modules may control the sending and receiving of data packets over networks. Security modules control access to all data and modules. All or part of the system and process can be implemented as a computer program product, tangibly embodied in an information carrier, such as a machine-readable storage device or in a propagated signal, for execution by, or to control the operation of, data processing apparatus, such as a programmable processor, computer, or deployed to be executed on multiple computers at one site or distributed across multiple sites. Memory storage may also include multiple distributed memory units, including one or more types of storage media.

Method steps associated with the system and process can be rearranged and/or one or more such steps can be omitted to achieve the same, or similar, results to those described herein. It is to be understood that the embodiments described hereinabove are cited by way of example, and that the present invention is not limited to what has been particularly shown and described hereinabove. Rather, the scope of the present invention includes variations and modifications thereof which would occur to persons skilled in the art upon reading the foregoing description and which are not disclosed in the prior art.

1. A computer-based method for screening DNA sequences to detect obfuscated sequences of concern (SOCs), comprising:
receiving a query sequence for screening against a target database of sequences;
applying a first alignment algorithm to generate multiple local alignments of the query sequence with a target sequence of the target database, wherein each local alignment aligns a substring of the query sequence to a substring of the target sequence to maximize an alignment score;
from the aligned substrings of the query sequence, determining unaligned sections of the query sequence to be alignment gaps; and
removing a number of largest alignment gaps of the query sequence to generate a clean query sequence and a respective clean alignment score that is an indicator of a homology of the target sequence and an obfuscated SOC in the query sequence.

2. The method of claim 1, wherein the number of largest alignment gaps is determined as one less than a number of local alignments of the multiple local alignments that, when combined, align with more of the target sequence than any other combination of the multiple local alignments.

3. The method of claim 1, further comprising: applying a second alignment algorithm to generate a clean alignment between the clean query sequence and the target sequence and to generate the clean alignment score, and outputting the clean alignment score indicating the homology of the target sequence and the obfuscated SOC in the query sequence.

4. The method of claim 3, wherein the first and second alignment algorithms are the same alignment algorithm.

5. The method of claim 3, wherein the first and second alignment algorithms are BLAST algorithms.

6. The method of claim 3, further comprising: reducing the clean alignment score by a gap removal penalty proportional to the number of largest alignment gaps removed, to calculate an adjusted clean alignment score indicating the homology of the target sequence and the obfuscated SOC in the query sequence.

7. The method of claim 6, wherein the adjusted clean alignment score is further adjusted by addition of a probability of biologically successful gap removal to generate a physical SOC.

8. The method of claim 6, wherein the gap removal penalty proportional to the number of largest alignment gaps removed is a per gap removal penalty (prm) multiplied by the number of largest alignment gaps removed.

9. The method of claim 8, wherein prm is a function of a number of base pairs removeable by bioengineering tools.

10. The method of claim 9, wherein the adjusted clean alignment score further includes a negative increment for each gap opening (pgo) and for each gap extension (pgx), and wherein prm=pgo+pgx·x, where x is the removeable number of base pairs.

11. The method of claim 1, further comprising removing different numbers of alignment gaps from the query sequence to generate different clean query sequences; reapplying the second alignment algorithm to each of the different clean query sequences to generate multiple respective clean alignments and respective adjusted clean alignment scores; and wherein outputting the clean alignment and the adjusted clean alignment score comprises determining a maximum score from among the multiple adjusted clean alignment scores and outputting the maximum score as the adjusted clean alignment score and outputting the respective clean alignment.

12. The method of claim 11, further comprising iterating generation of clean alignments and respective adjusted clean alignment scores with respect to all of the target database sequences.

13. The method of claim 12, further comprising ordering the clean alignments and the adjusted clean alignment scores generated with respect to all of the target database sequences according to the adjusted clean alignment score.

14. The method of claim 1, wherein the database of target sequences is a database of SOCs.

15. The method of claim 1, where the number of alignment gaps removed is set to be all gaps greater than a preset threshold number of base pairs, and wherein the number of alignment gaps removed is subsequently incremented by an iterative process removing the largest alignment gap not previously removed, until the adjusted clean alignment score calculated following the gap removal does not increase.

16. The method of claim 1, wherein the first alignment score includes a positive increment for each matching character (rm) and a negative increment for each mismatching character (pmm), for each gap opening (pgo), and for each gap extension (pgx).
